(19) United States
(12) Patent Application Publication
(10) Pub. No.: US 2005/0288185 A1
(43) Pub. Date: Dec. 29, 2005

(54) Substituted phenyluracils

(76) Inventors: Hans-Georg Schwarz, Langenfeld (DE); Roland Andree, Langenfeld (DE); Dorothee Hoischen, Dusseldorf (DE); Karl-Heinz Linker, Leverkusen (DE); Mark Wilhelm Drewes, Langenfeld (DE); Peter Dahmen, Neuss (DE); Dieter Feucht, Monheim (DE); Rolf Pontzen, Leichlingen (DE)

Correspondence Address: BAYER CROPSCIENCE LP;Patent Department, 100 BAYER ROAD, PITTSBURGH, PA, 15205-9741, US

(21) Appl. No.: 10/515,516

(22) PCT Filed: May 16, 2003
(86) PCT No.: PCT/EP03/05160

§ 371 Date: Jul. 11, 2005

(30) Foreign Application Priority Data
May 29, 2002 (DE) 102 23 914.2

(51) Int. Cl. A01N 43/54; C07D 45/02; C07D 43/02

(52) U.S. Cl. 504/243; 544/309; 544/310

(57) ABSTRACT

The invention relates to novel substituted phenyluracils of the formula (I)

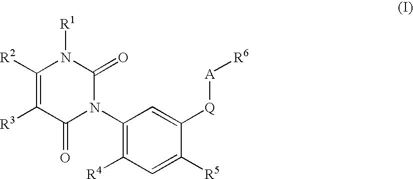

in which A, Q, R¹, R², R³, R⁴, R⁵ and R⁶ are as defined in the disclosure, to processes for their preparation and to their use as crop treatment agents.

The invention relates to novel substituted phenyluracils, to processes for their preparation and to their use as crop treatment agents, in particular as herbicides.

Certain substituted phenyluracils with a structure similar to that of the compounds of the present invention are already known (for example EP-A-255 047, EP-A-831 091, EP-A-1 061 075, U.S. Pat. No. 6,207,830). However, these compounds have hitherto not attained any particular importance since they have various disadvantages.

This invention now provides novel substituted phenyluracils of the general formula (I) 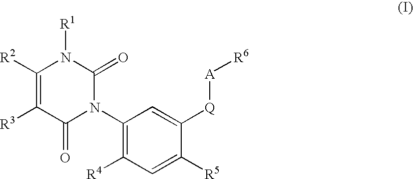
in which
A represents alkanediyl having 1 to 6 carbon atoms, alkenediyl having 2 to 6 carbon atoms or alkynediyl having 2 to 6 carbon atoms,
Q represents O (oxygen), S (sulphur), SO or SO₂,
R¹ represents hydrogen, amino or optionally cyano-, halogen- or C₁-C₄-alkoxy-substituted alkyl having 1 to 6 carbon atoms,
R² represents carboxyl, cyano, carbamoyl, thiocarbamoyl or in each case optionally cyano-, halogen- or C₁-C₄-alkoxy-substituted alkyl or alkoxycarbonyl having in each case 1 to 6 carbon atoms in the alkyl groups,
R³ represents hydrogen, halogen or optionally halogen-substituted alkyl having 1 to 6 carbon atoms,
R⁴ represents hydrogen, cyano, carbamoyl, thiocarbamoyl or halogen,
R⁵ represents nitro, cyano, carbamoyl, thiocarbamoyl, halogen or in each case optionally halogen-substituted alkyl or alkoxy having in each case 1 to 6 carbon atoms, and
R⁶ represents monocyclic or bicyclic heterocyclyl having up to 9 carbon atoms and up to 5 heteroatoms selected from up to 5 nitrogen atoms and/or up to 2 oxygen atoms and/or up to 2 sulphur atoms and also optionally additionally up to 2 SO groups, up to 2 SO₂ groups, up to 2 CO groups or up to 2 CS groups, which heterocyclyl is attached via a carbon atom to A and is substituted by cyano, carboxyl, carbamoyl or thiocarbamoyl, or by C₁-C₆-alkoxycarbonyl, C₃-C₆-cycloalkyloxycarbonyl or C₁-C₆-alkylaminocarbonyl (which are in each case optionally substituted by cyano, carboxyl, halogen or C₁-C₆-alkoxycarbonyl), or by di-(C₁-C₄-alkyl)aminocarbonyl or N—(C₁-C₄-alkoxy)-C₁-C₄-alkylaminocarbonyl, or by C₂-C₆-alkenyloxycarbonyl or C₂-C₆-alkynyloxycarbonyl (which are in each case optionally substituted by halogen), or by cyano-C₁-C₆-alkyl, carboxy-C₁-C₆-alkyl, C₁-C₄-alkoxycarbonyl-C₁-C₆-alkyl, cyano-C₂-C₆-alkenyl, carboxy-C₂-C₆-alkenyl or C₁-C₄-alkoxycarbonyl-C₂-C₆-alkenyl (which are in each case optionally substituted by halogen), and optionally additionally by nitro, halogen or by C₁-C₄-alkyl, C₁-C₄-alkoxy, C₁-C₄-alkylthio, C₁-C₄-alkylsulphinyl or C₁-C₄-alkylsulphonyl (which are in each case optionally substituted by halogen), including the possible stereoisomeric forms.

In the definitions, the hydrocarbon chains, such as alkyl or alkenyl, are in each case straight-chain or branched—including in combination with heteroatoms, such as in alkoxy.

Optionally substituted radicals can be mono- or polysubstituted, and in the case of polysubstitution the substituents can be identical or different.

Preferred substituents or ranges of the radicals present in the formulae given above and below are defined as below:
A preferably represents alkanediyl having 1 to 5 carbon atoms, alkenediyl having 2 to 5 carbon atoms or alkynediyl having 2 to 5 carbon atoms.
Q preferably represents O (oxygen), S (sulphur) or SO₂.
R¹ preferably represents hydrogen, amino or optionally cyano-, halogen- or C₁-C₄-alkoxy-substituted alkyl having 1 to 4 carbon atoms.
R² preferably represents carboxyl, cyano, carbamoyl, thiocarbamoyl or in each case optionally cyano-, halogen- or C₁-C₄-alkoxy-substituted alkyl or alkoxy-carbonyl having in each case 1 to 4 carbon atoms in the alkyl groups.
R³ preferably represents hydrogen, halogen or optionally halogen-substituted alkyl having 1 to 4 carbon atoms.
R⁴ preferably represents hydrogen, cyano, carbamoyl, thiocarbamoyl, fluorine, chlorine, bromine or iodine.
R⁵ preferably represents nitro, cyano, carbamoyl, thiocarbamoyl, halogen or in each case optionally halogen-substituted alkyl or alkoxy having in each case 1 to 4 carbon atoms.
R⁶ preferably represents monocyclic or bicyclic heterocyclyl having up to 9 carbon atoms and up to 5 heteroatoms selected from up to 3 nitrogen atoms and/or one oxygen atom and/or one sulphur atom, and also optionally additionally one —SO group, one —SO₂ group, one —CO group or one —CS group, which heterocyclyl is attached via a carbon atom to A and is substituted by cyano, carboxyl, carbamoyl or thiocarbamoyl, or by C₁-C₅-alkoxycarbonyl, C₅-C₆-cycloalkyloxycarbonyl or C₁-C₅-alkylaminocarbonyl (which are in each case optionally substituted by cyano, carboxyl, halogen or C₁-C₄-alkoxycarbonyl), or by di-(C₁-C₄-alkyl)aminocarbonyl or N—(C₁-C₄-alkoxy)-C₁-C₄-alkylaminocarbonyl, or by C₂-C₅-alkenyloxycarbonyl or C₂-C₅-alkynyloxycarbonyl (which are in each case optionally substituted by halogen), or by cyano-C₁-C₅-alkyl, carboxy-C₁-C₅-alkyl, C₁-C₄-alkoxy-carbonyl-C₁-C₅-alkyl, cyano-C₂-C₅-alkenyl, carboxy-C₂-C₅-alkenyl or C₁-C₄-alkoxycarbonyl-C₂-C₅-alkenyl (which are in each case optionally substituted by halogen), and optionally additionally by nitro, halogen or by C₁-C₄-alkyl, C₁-C₄-alkoxy, C₁-C₄-alkylthio, C₁-C₄-alkylsulphinyl or C₁-C₄-alkylsulphonyl (which are in each case optionally substituted by halogen).
A particularly preferably represents alkanediyl having 1 to 4 carbon atoms, alkenediyl having 2 to 4 carbon atoms or alkynediyl having 2 to 4 carbon atoms.
Q particularly preferably represents O (oxygen) or S (sulphur).
R¹ particularly preferably represents hydrogen, amino or optionally cyano-, fluorine-, chlorine-, methoxy- or ethoxy-substituted methyl, ethyl or n- or i-propyl.
R² particularly preferably represents carboxyl, cyano, carbamoyl, thiocarbamoyl or in each case optionally cyano-, fluorine-, chlorine-, methoxy-, ethoxy-, n- or i-propoxy-substituted alkyl or alkoxycarbonyl having in each case 1 to 3 carbon atoms in the alkyl groups.
R³ particularly preferably represents hydrogen, fluorine, chlorine, bromine or optionally fluorine- and/or chlorine-substituted methyl, ethyl or n- or i-propyl.
R⁴ particularly preferably represents hydrogen, cyano, carbamoyl, thiocarbamoyl, fluorine, chlorine or bromine.
R⁵ particularly preferably represents nitro, cyano, carbamoyl, thiocarbamoyl, fluorine, chlorine, bromine or in each case optionally fluorine- and/or chlorine-substituted alkyl or alkoxy having in each case 1 to 3 carbon atoms.
R⁶ particularly preferably represents heterocyclyl from the group consisting of furyl, benzofuryl, tetrahydrofuryl, thienyl, benzothienyl, pyrrolyl, benzopyrrolyl, pyrrolinyl, pyrazolyl, benzopyrazolyl, pyrazolinyl, imidazolyl, benzimidazolyl, imidazolinyl, oxazolyl, benzoxazolyl, oxazolinyl, isoxazolyl, isoxazolinyl, thiazolyl, benzthiazolyl, thiazolinyl, thiadiazolyl, triazolyl, pyridinyl, pyrimidinyl, triazinyl, which is attached via a carbon atom to A and is substituted by cyano, carboxyl, carbamoyl or thiocarbamoyl, or by C₁-C₄-alkoxycarbonyl, C₅-C₆-cycloalkyloxycarbonyl or C₁-C₄-alkylaminocarbonyl (which are in each case optionally substituted by cyano, carboxyl, halogen or C₁-C₄-alkoxycarbonyl), or by di-(C₁-C₃-alkyl)aminocarbonyl or N—(C₁-C₃-alkoxy)-C₁-C₃-alkylaminocarbonyl, or by C₃-C₄-alkenyloxycarbonyl or C₃-C₄-alkynyloxycarbonyl (which are in each case optionally substituted by halogen), or by cyano-C₁-C₃-alkyl, carboxy-C₁-C₃-alkyl, C₁-C₄-alkoxycarbonyl-C₁-C₃-alkyl, cyano-C₂-C₃-alkenyl, carboxy-C₂-C₃-alkenyl or C₁-C₄-alkoxycarbonyl-C₂-C₃-alkenyl (which are in each case optionally substituted by halogen), and optionally additionally by nitro, halogen or by C₁-C₄-alkyl, C₁-C₄-alkoxy, C₁-C₄-alkylthio, C₁-C₄-alkylsulphinyl or C₁-C₄-alkylsulphonyl (which are in each case optionally substituted by halogen).
A very particularly preferably represents methylene, ethane-1,1-diyl, ethane-1,2-diyl(dimethylene), propane-1,1-diyl, propane-1,2-diyl, propane-1,3-diyl, ethene-1,1-diyl, ethene-1,2-diyl, propene-1,1-diyl, propene-1,2-diyl, propene-1,3-diyl, ethyne-1,2-diyl or propyne-1,3-diyl.
Q very particularly preferably represents O (oxygen).
R¹ very particularly preferably represents hydrogen, amino or in each case optionally cyano-, fluorine-, chlorine-, methoxy- or ethoxy-substituted methyl or ethyl.
R² very particularly preferably represents carboxyl, cyano, carbamoyl, thiocarbamoyl or in each case optionally cyano-, fluorine-, chlorine-, methoxy- or ethoxy-substituted methyl, ethyl, methoxycarbonyl or ethoxycarbonyl.
R³ very particularly preferably represents hydrogen, fluorine, chlorine, bromine or in each case optionally fluorine- and/or chlorine-substituted methyl or ethyl.
R⁴ very particularly preferably represents hydrogen, cyano, fluorine or chlorine.
R⁵ very particularly preferably represents nitro, cyano, carbamoyl, thiocarbamoyl, fluorine, chlorine, bromine or in each case optionally fluorine- and/or chlorine-substituted methyl, ethyl, methoxy or ethoxy.
R⁶ very particularly preferably represents heterocyclyl from the group consisting of furyl, thienyl, pyrrolyl, pyrrolinyl, pyrazolyl, pyrazolinyl, imidazolyl, imidazolinyl, oxazolyl, oxazolinyl, isoxazolyl, isoxazolinyl, thiazolyl, thiazolinyl, pyridinyl, pyrimidinyl which is attached via a carbon atom to A and is substituted by cyano, carboxyl, carbamoyl or thiocarbamoyl, or by methoxycarbonyl, ethoxycarbonyl, n- or i-propoxycarbonyl, cyclopentyloxycarbonyl, cyclohexyloxycarbonyl, methylaminocarbonyl, ethylaminocarbonyl, n- or i-propylaminocarbonyl (which are in each case optionally substituted by cyano, carboxyl, fluorine, chlorine, methoxycarbonyl, ethoxycarbonyl, n- or i-propoxycarbonyl), or by dimethylaminocarbonyl, diethylaminocarbonyl or N-methoxymethylaminocarbonyl, or by propenyloxycarbonyl, butenyloxycarbonyl, propynyloxycarbonyl or butynyloxycarbonyl (which are in each case optionally substituted by fluorine or chlorine), or by cyanomethyl, cyanoethyl, cyanopropyl, carboxymethyl, carboxyethyl, carboxypropyl, methoxycarbonylmethyl, ethoxycarbonylmethyl, n- or i-propoxycarbonylmethyl, methoxycarbonylethyl, ethoxycarbonylethyl, n- or i-propoxycarbonylethyl, cyanoethenyl, cyanopropenyl, carboxyethenyl, carboxypropenyl, methoxycarbonylethenyl, ethoxycarbonylethenyl, n- or i-propoxycarbonylethenyl, methoxycarbonylpropenyl, ethoxycarbonylpropenyl, n- or i-propoxycarbonylpropenyl (which are in each case optionally substituted by fluorine and/or chlorine), and optionally additionally by nitro, fluorine, chlorine, bromine, methyl, ethyl, n- or i-propyl, n-, i-, s- or t-butyl, methoxy, ethoxy, n- or i-propoxy, methylthio, ethylthio, n- or i-propylthio, methylsulphinyl, ethylsulphinyl, methylsulphonyl or ethylsulphonyl (which are in each case optionally substituted by fluorine and/or chlorine).
A most preferably represents methylene.
R¹ most preferably represents hydrogen, methyl or amino.
R² most preferably represents trifluoromethyl.
R³ most preferably represents hydrogen.
R⁴ most preferably represents fluorine.
R⁵ most preferably represents chlorine, bromine or cyano.
R⁶ most preferably represents one of the groupings (A) to (L) below 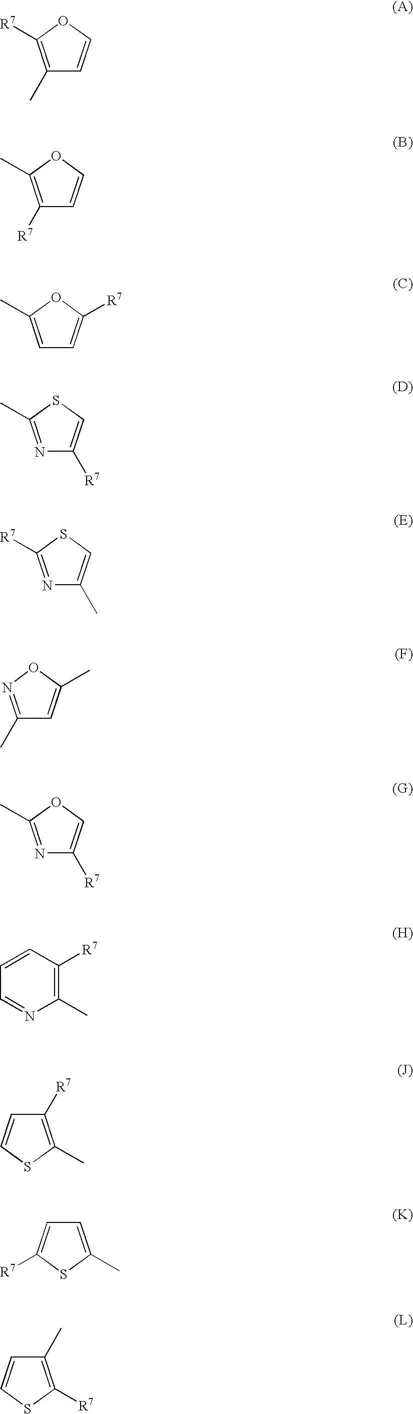
   in which
R⁷ represents carboxyl, carbamoyl, methoxycarbonyl, ethoxycarbonyl or n- or i-propoxycarbonyl.
R⁷ preferably represents methoxycarbonyl or ethoxycarbonyl.

Preference according to the invention is given to the compounds of the formula (I) which contain a combination of the meanings given above as being preferred. Particular preference according to the invention is given to the compounds of the formula (I) which contain a combination of the meanings given above as being particularly preferred.

Very particular preference according to the invention is given to the compounds of the formula (I) which contain a combination of the meanings given above as being very particularly preferred.

Most preference according to the invention is given to the compounds of the formula (I) which contain a combination of the meanings given above as being most preferred.

A very particularly preferred group are those compounds of the formula (I) in which
A represents methylene,
Q represents O (oxygen),
R¹ represents hydrogen, amino or methyl,
R² represents cyano or trifluoromethyl,
R³ represents hydrogen, chlorine, bromine or methyl,
R⁴ represents hydrogen, fluorine or chlorine,
R⁵ represents cyano, thiocarbamoyl, chlorine, bromine or trifluoromethyl,
R⁶ represents furyl which is substituted by cyano, carboxyl, carbamoyl, thiocarbamoyl, by methoxycarbonyl, ethoxycarbonyl, n- or i-propoxycarbonyl, and optionally additionally by nitro, fluorine, chlorine, bromine, methyl, ethyl, methoxy, ethoxy, methylthio, ethylthio, methylsulphinyl, ethylsulphinyl, methylsulphonyl or ethylsulphonyl.

A further very particularly preferred group are those compounds of the formula (I) in which
A represents methylene,
Q represents O (oxygen),
R¹ represents hydrogen, amino or methyl,
R² represents cyano or trifluoromethyl,
R³ represents hydrogen, chlorine, bromine or methyl,
R⁴ represents hydrogen, fluorine or chlorine,
R⁵ represents cyano, thiocarbamoyl, chlorine, bromine or trifluoromethyl,
R⁶ represents oxazolyl which is substituted by cyano, carboxyl, carbamoyl, thiocarbamoyl, by methoxycarbonyl, ethoxycarbonyl, n- or i-propoxycarbonyl, and optionally additionally by nitro, fluorine, chlorine, bromine, methyl, ethyl, methoxy, ethoxy, methylthio, ethylthio, methylsulphinyl, ethylsulphinyl, methylsulphonyl or ethylsulphonyl.

A further very particularly preferred group are those compounds of the formula (I) in which
A represents methylene,
Q represents O (oxygen),
R¹ represents hydrogen, amino or methyl,
R² represents cyano or trifluoromethyl,
R³ represents hydrogen, chlorine, bromine or methyl,
R⁴ represents hydrogen, fluorine or chlorine,
R⁵ represents cyano, thiocarbamoyl, chlorine, bromine or trifluoromethyl,
R⁶ represents isoxazolyl which is substituted by cyano, carboxyl, carbamoyl, thiocarbamoyl, by methoxycarbonyl, ethoxycarbonyl, n- or i-propoxycarbonyl, and optionally additionally by nitro, fluorine, chlorine, bromine, methyl, ethyl, methoxy, ethoxy, methylthio, ethylthio, methylsulphinyl, ethylsulphinyl, methylsulphonyl or ethylsulphonyl.

A further very particularly preferred group are those compounds of the formula (I) in which
A represents methylene,
Q represents O (oxygen),
R¹ represents hydrogen, amino or methyl,
R² represents cyano or trifluoromethyl,
R³ represents hydrogen, chlorine, bromine or methyl,
R⁴ represents hydrogen, fluorine or chlorine,
R⁵ represents cyano, thiocarbamoyl, chlorine, bromine or trifluoromethyl,
R⁶ represents thiazolyl which is substituted by cyano, carboxyl, carbamoyl, thiocarbamoyl, by methoxycarbonyl, ethoxycarbonyl, n- or i-propoxycarbonyl, and optionally additionally by nitro, fluorine, chlorine, bromine, methyl, ethyl, methoxy, ethoxy, methylthio, ethylthio, methylsulphinyl, ethylsulphinyl, methylsulphonyl or ethylsulphonyl.

A further very particularly preferred group are those compounds of the formula (I) in which
A represents methylene,
Q represents O (oxygen),
R¹ represents hydrogen, amino or methyl,
R² represents cyano or trifluoromethyl,
R³ represents hydrogen, chlorine, bromine or methyl,
R⁴ represents hydrogen, fluorine or chlorine,
R⁵ represents cyano, thiocarbamoyl, chlorine, bromine or trifluoromethyl,
R⁶ represents pyridinyl which is substituted by cyano, carboxyl, carbamoyl, thiocarbamoyl, by methoxycarbonyl, ethoxycarbonyl, n- or i-propoxycarbonyl, and optionally additionally by nitro, fluorine, chlorine, bromine, methyl, ethyl, methoxy, ethoxy, methylthio, ethylthio, methylsulphinyl, ethylsulphinyl, methylsulphonyl or ethylsulphonyl.

A further very particularly preferred group are those compounds of the formula (I) in which
A represents methylene,
Q represents O (oxygen),
R¹ represents hydrogen, amino or methyl,
R² represents cyano or trifluoromethyl,
R³ represents hydrogen, chlorine, bromine or methyl,
R⁴ represents hydrogen, fluorine or chlorine,
R⁵ represents cyano, thiocarbamoyl, chlorine, bromine or trifluoromethyl,
R⁶ represents thienyl which is substituted by cyano, carboxyl, carbamoyl, thiocarbamoyl, by methoxycarbonyl, ethoxycarbonyl, n- or i-propoxycarbonyl, and optionally additionally by nitro, fluorine, chlorine, bromine, methyl, ethyl, methoxy, ethoxy, methylthio, ethylthio, methylsulphinyl, ethylsulphinyl, methylsulphonyl or ethylsulphonyl.

The general or preferred radical definitions given above apply both to the end products of the formula (I) and, correspondingly, to the starting materials or intermediates required in each case for the preparation. These radical definitions can be combined with one another as desired, i.e. including combinations between the given preferred ranges.

The novel substituted phenyluracils of the general formula (I) have strong and selective herbicidal activity.

The novel substituted phenyluracils of the general formula (I) are obtained when hydroxyphenyl- or mercaptophenyluracils of the general formula (II) 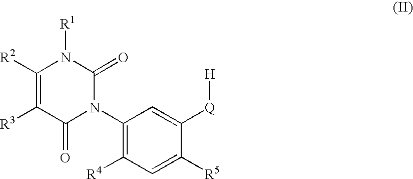
in which
Q, R¹, R², R³, R⁴ and R⁵ are as defined above
are reacted with substituted heterocycles of the general formula (III) 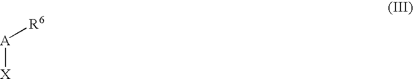
   in which
A and R⁶ are as defined above and
X represents halogen or represents in each case optionally substituted alkylsulphonyloxy or arylsulphonyloxy,
if appropriate in the presence of one or more reaction auxiliaries and if appropriate in the presence of one or more diluents,
and the resulting compounds of the formula (I) are, if appropriate, converted by customary methods into other compounds of the formula (I).

Using, for example, 3-(2,4-dichloro-5-mercaptophenyl)-1-methyl-6-trifluoromethyl-1H-pyrimidine-2,4-dione and methyl 2-bromomethylfuran-4-carboxylate as starting materials, the course of the reaction in the process according to the invention can be illustrated by the formula scheme below:

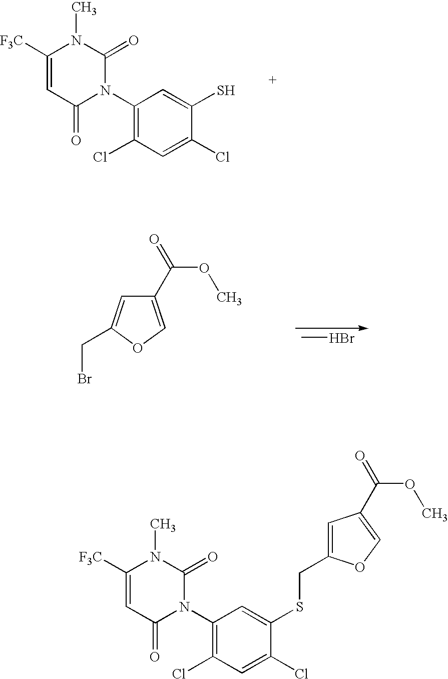

The formula (II) provides a general definition of the hydroxyphenyl- and mercaptophenyluracils to be used as starting materials in the process according to the invention for preparing compounds of the formula (I). In the formula (II), Q, R¹, R², R³, R⁴ and R⁵ preferably have those meanings which have already been mentioned above, in connection with the description of the compounds of the formula (I) according to the invention, as being preferred, particularly preferred, very particularly preferred or most preferred for Q, R¹, R², R³, R⁴ and R⁵.

The starting materials of the general formula (II) are known and/or can be prepared by processes known per se (cf. WO 97/01541, WO 98/54155).

The formula (III) provides a general definition of the substituted heterocycles further to be used as starting materials in the process according to the invention for preparing compounds of the formula (I). In the formula (III), A and R⁶ preferably have those meanings which have already been mentioned above, in connection with the description of the compounds of the formula (I) according to the invention, as being preferred, particularly preferred, very particularly preferred or most preferred for A and R⁶; X preferably represents fluorine, chlorine, bromine, iodine, optionally fluorine- and/or chlorine-substituted C₁-C₄-alkylsulphonyloxy or optionally fluorine-, chlorine- or methyl-substituted phenylsulphonyloxy, in particular chlorine, bromine, methylsulphonyloxy, phenylsulphonyloxy or tolylsulphonyloxy.

The starting materials of the general formula (III) are known and/or can be prepared by processes known per se (cf. J. Chem. Soc., Chem. Commun. 19 (1996), 2251-2252; J. Med. Chem. 38 (1995), 4806-4820; Tetrahedron 54 (1998), 7525-7538; Dokl. Akad. Nauk. Arm. SSR 17 (1953), 97-103; Organic Process Research & Development 5 (2001), 37-44).

The substituted heterocycles of the general formula (III) are obtained when, for example, alkylheterocycles of the general formula (IV) 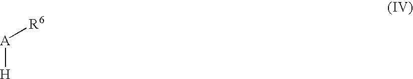
in which
A and R⁶ are as defined above
are reacted with halogenating agents, such as, for example, N-bromosuccinimide or N-chlorosuccinimide, preferably in the presence of a reaction auxiliary, such as, for example, 2,2′-azobis-2-methylpropanenitrile, and preferably in the presence of a diluent, such as, for example, carbon tetrachloride, at temperatures between 0° C. and 100° C. (cf. the Preparation Examples).

The substituted heterocycles of the general formula (IV) are known organic chemicals for synthesis.

The process according to the invention for preparing the compounds of the general formula (I) is preferably carried out using a reaction auxiliary. Suitable reaction auxiliaries for the process according to the invention are, in general, the customary inorganic or organic bases or acid acceptors. These preferably include alkali metal or alkaline earth metal acetates, amides, carbonates, bicarbonates, hydrides, hydroxides or alkoxides, such as, for example, sodium acetate, potassium acetate or calcium acetate, lithium amide, sodium amide, potassium amide or calcium amide, sodium carbonate, potassium carbonate or calcium carbonate, sodium bicarbonate, potassium bicarbonate or calcium bicarbonate, lithium hydride, sodium hydride, potassium hydride or calcium hydride, lithium hydroxide, sodium hydroxide, potassium hydroxide or calcium hydroxide, sodium methoxide, ethoxide, n- or i-propoxide, n-, i-, s- or -t-butoxide or potassium methoxide, ethoxide, n- or i-propoxide, n-, i-, s- or t-butoxide; furthermore also basic organic nitrogen compounds, such as, for example, trimethylamine, triethylamine, tripropylamine, tributylamine, ethyl-diisopropylamine, N,N-dimethylcyclohexylamine, dicyclohexylamine, ethyl-dicyclohexylamine, N,N-dimethylaniline, N,N-dimethylbenzylamine, pyridine, 2-methyl-, 3-methyl-, 4-methyl-, 2,4-dimethyl-, 2,6-dimethyl-, 3,4-dimethyl- and 3,5-dimethylpyridine, 5-ethyl-2-methylpyridine, 4-dimethylaminopyridine, N-methylpiperidine, 1,4-diaza-bicyclo[2.2.2]octane (DABCO), 1,5-diazabicyclo[4.3.0]non-5-ene (DBN) or 1,8-diazabicyclo[5.4.0]undec-7-ene (DBU).

The process according to the invention for preparing the compounds of the general formula (I) is preferably carried out using a diluent. Suitable diluents for carrying out the process according to the invention are especially inert organic solvents. These include, in particular, aliphatic, alicyclic or aromatic, optionally halogenated hydrocarbons, such as, for example, benzine, benzene, toluene, xylene, chlorobenzene, dichlorobenzene, petroleum ether, hexane, cyclohexane, dichloro-methane, chloroform, carbon tetrachloride; ethers, such as diethyl ether, diisopropyl ether, dioxane, tetrahydrofuran or ethylene glycol dimethyl ether or ethylene glycol diethyl ether; ketones, such as acetone, butanone or methyl isobutyl ketone; nitriles, such as acetonitrile, propionitrile or butyronitrile; amides, such as N,N-dimethylformamide, N,N-dimethylacetamide, N-methylformanilide, N-methylpyrrolidone or hexamethylphosphoric triamide; esters, such as methyl acetate or ethyl acetate; sulphoxides, such as dimethylsulphoxide, alcohols, such as methanol, ethanol, n- or i-propanol, ethylene glycol monomethyl ether, ethylene glycol monoethyl ether, diethylene glycol monomethyl ether, diethylene glycol monoethyl ether, mixtures thereof with water or pure water.

When carrying out the process according to the invention, the reaction temperatures can be varied within a relatively wide range. In general, the process is carried out at temperatures between 0° C. and 150° C., preferably between 10° C. and 120° C.

The process according to the invention is generally carried out under atmospheric pressure. However, it is also possible to carry out the process according to the invention under elevated or reduced pressure—in general between 0.1 bar and 10 bar.

For carrying out the process according to the invention, the starting materials are generally employed in approximately equimolar amounts. However, it is also possible to use a relatively large excess of one of the components. The reaction is generally carried out in a suitable diluent in the presence of a reaction auxiliary, and the reaction mixture is generally stirred at the required temperature for a number of hours. Work-up is carried out by customary methods (cf. the Preparation Examples).

The compounds of the general formula (I) can be converted by customary methods into other compounds of the general formula (I) in accordance with the above definition, for example compounds in which R¹ represents hydrogen can be converted into corresponding compounds in which R¹ represents amino by reaction with suitable aminating agents, such as, for example, 1-aminooxy-2,4-dinitrobenzene (cf. Preparation Example) or into corresponding compounds in which R¹ represents methyl by reaction with dimethyl sulphate or methyl bromide (cf. Preparation Example).

The active compounds according to the invention can be used as defoliants, desiccants, haulm killers and, especially, as weed killers. Weeds in the broadest sense are understood to mean all plants which grow in locations where they are undesired. Whether the substances according to the invention act as total or selective herbicides depends essentially on the amount used.

The active compounds according to the invention can be used, for example, in connection with the following plants:

Dicotyledonous weeds of the genera: Abutilon, Amaranthus, Ambrosia, Anoda, Anthemis, Aphanes, Atriplex, Bellis, Bidens, Capsella, Carduus, Cassia, Centaurea, Chenopodium, Cirsium, Convolvulus, Datura, Desmodium, Emex, Erysimum, Euphorbia, Galeopsis, Galinsoga, Galium, Hibiscus, Ipomoea, Kochia, Lamium, Lepidium, Lindernia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Urtica, Veronica, Viola, Xanthium.

Dicotyledonous crops of the genera: Arachis, Beta, Brassica, Cucumis, Cucurbita, Helianthus, Daucus, Glycine, Gossypium, Ipomoea, Lactuca, Linum, Lycopersicon, Nicotiana, Phaseolus, Pisum, Solanum, Vicia.

Monocotyledonous weeds of the genera: Aegilops, Agropyron, Agrostis, Alopecurus, Apera, Avena, Brachiaria, Bromus, Cenchrus, Commelina, Cynodon, Cyperus, Dactyloctenium, Digitaria, Echinochloa, Eleocharis, Eleusine, Eragrostis, Eriochloa, Festuca, Fimbristylis, Heteranthera, Imperata, Ischaemum, Leptochloa, Lolium, Monochoria, Panicum, Paspalum, Phalaris, Phleum, Poa, Rottboellia, Sagittaria, Scirpus, Setaria, Sorghum.

Monocotyledonous crops of the genera: Allium, Ananas, Asparagus, Avena, Hordeum, Oryza, Panicum, Saccharum, Secale, Sorghum, Triticale, Triticum, Zea.

However, the use of the active compounds according to the invention is in no way restricted to these genera, but also extends in the same manner to other plants.

The active compounds according to the invention are suitable, depending on the concentration, for the total control of weeds, for example on industrial terrain and rail tracks, and on paths and areas with and without tree plantings. Similarly, the active compounds according to the invention can be employed for controlling weeds in perennial crops, for example forests, decorative tree plantings, orchards, vineyards, citrus groves, nut orchards, banana plantations, coffee plantations, tea plantations, rubber plantations, oil palm plantations, cocoa plantations, soft fruit plantings and hop fields, on lawns, turf and pastureland, and for the selective control of weeds in annual crops.

The compounds of the formula (I) according to the invention have strong herbicidal activity and a broad active spectrum when used on the soil and on above-ground parts of plants. To a certain extent they are also suitable for the selective control of monocotyledonous and dicotyledonous weeds in monocotyledonous and dicotyledonous crops, both by the pre-emergence and by the post-emergence method.

At certain concentrations or application rates, the active compounds according to the invention can also be employed for controlling animal pests and fungal or bacterial plant diseases. If appropriate, they can also be used as intermediates or precursors for the synthesis of other active compounds.

According to the invention, it is possible to treat all plants and parts of plants. Plants are to be understood here as meaning all plants and plant populations such as desired and undesired wild plants or crop plants (including naturally occurring crop plants). Crop plants can be plants which can be obtained by conventional breeding and optimization methods or by biotechnological and genetic engineering methods or combinations of these methods, including the transgenic plants and including plant cultivars which can or cannot be protected by plant breeders' certificates. Parts of plants are to be understood as meaning all above-ground and below-ground parts and organs of plants, such as shoot, leaf, flower and root, examples which may be mentioned being leaves, needles, stems, trunks, flowers, fruit-bodies, fruits and seeds and also roots, tubers and rhizomes. Parts of plants also include harvested plants and vegetative and generative propagation material, for example seedlings, tubers, rhizomes, cuttings and seeds.

The treatment of the plants and parts of plants according to the invention with the active compounds is carried out directly or by action on their environment, habitat or storage area according to customary treatment methods, for example by dipping, spraying, evaporating, atomizing, broadcasting, brushing-on and, in the case of propagation material, in particular in the case of seeds, furthermore by one- or multi-layer coating.

The active compounds can be converted into the customary formulations, such as solutions, emulsions, wettable powders, suspensions, powders, dusts, pastes, soluble powders, granules, suspo-emulsion concentrates, natural and synthetic substances impregnated with active compound, and microencapsulations in polymeric substances.

These formulations are produced in a known manner, for example by mixing the active compounds with extenders, that is to say liquid solvents and/or solid carriers, optionally with the use of surfactants, that is to say emulsifiers and/or dispersants and/or foam-formers.

If the extender used is water, it is also possible to use, for example, organic solvents as auxiliary solvents. Liquid solvents which are mainly suitable are: aromatics, such as xylene, toluene or alkylnaphthalenes, chlorinated aromatics and chlorinated aliphatic hydrocarbons, such as chlorobenzenes, chloroethylenes or methylene chloride, aliphatic hydrocarbons, such as cyclohexane or paraffins, for example petroleum fractions, mineral and vegetable oils, alcohols, such as butanol or glycol, and also their ethers and esters, ketones, such as acetone, methyl ethyl ketone, methyl isobutyl ketone or cyclohexanone, strongly polar solvents, such as dimethylformamide and dimethyl sulphoxide, and water.

Suitable solid carriers are: for example ammonium salts and ground natural minerals, such as kaolins, clays, talc, chalk, quartz, attapulgite, montmorillonite or diato-maceous earth, and ground synthetic minerals, such as finely divided silica, alumina and silicates; suitable solid carriers for granules are: for example crushed and fractionated natural rocks, such as calcite, marble, pumice, sepiolite, dolomite and synthetic granules of inorganic and organic meals, and granules of organic material, such as sawdust, coconut shells, maize cobs and tobacco stalks; suitable emulsifiers and/or foam formers are: for example nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates and protein hydrolysates; suitable dispersants are: for example lignosulphite waste liquors and methylcellulose.

Tackifiers, such as carboxymethylcellulose, natural and synthetic polymers in the form of powders, granules or latices, such as gum arabic, polyvinyl alcohol and polyvinyl acetate, and also natural phospholipids, such as cephalins and lecithins, and synthetic phospholipids can be used in the formulations. Other possible additives are mineral and vegetable oils.

It is possible to use colorants, such as inorganic pigments, for example iron oxide, titanium oxide, Prussian blue, and organic dyestuffs, such as alizarin dyestuffs, azo dyestuffs and metal phthalocyanine dyestuffs, and trace nutrients, such as salts of iron, manganese, boron, copper, cobalt, molybdenum and zinc.

The formulations generally comprise between 0.1 and 95 per cent by weight of active compound, preferably between 0.5 and 90%.

For controlling weeds, the active compounds according to the invention, as such or in their formulations, can also be used as mixtures with known herbicides and/or substances which improve the compatibility with crop plants (“safeners”), finished formulations or tank mixes being possible. Also possible are mixtures with weed-killers comprising one or more known herbicides and a safener.

Possible components for the mixtures are known herbicides, for example acetochlor, acifluorfen (-sodium), aclonifen, alachlor, alloxydim (-sodium), ametryne, amicarbazone, amidochlor, amidosulphuron, anilofos, asulam, atrazine, azafenidin, azimsulphuron, beflubutamid, benazolin (-ethyl), benfuresate, bensulphuron (-methyl), bentazon, benzfendizone, benzobicyclon, benzofenap, benzoylprop (-ethyl), bialaphos, bifenox, bispyribac (-sodium), bromobutide, bromofenoxim, bromoxynil, butachlor, butafenacil (-allyl), butroxydim, butylate, cafenstrole, caloxydim, carbetamide, carfentrazone (-ethyl), chlomethoxyfen, chlorarnben, chloridazon, chlorimuron (-ethyl), chlornitrofen, chlorsulphuron, chlortoluron, cinidon (-ethyl), cinmethylin, cinosulphuron, clefoxydim, clethodim, clodinafop (-propargyl), clomazone, clomeprop, clopyralid, clopyrasuiphuron (-methyl), cloransulam (-methyl), cumyluron, cyanazine, cybutryne, cycloate, cyclosulphamuron, cycloxydim, cyhalofop (-butyl), 2,4-D, 2,4-DB, desmedipham, diallate, dicamba, dichlorprop (—P), diclofop (-methyl), diclosulam, diethatyl (-ethyl), difenzoquat, diflufenican, diflufenzopyr, dimefuron, dimepiperate, dimethachlor, dimethametryn, dimethenamid, dimexyflam, dinitramine, diphenamid, diquat, dithiopyr, diuron, dymron, epropodan, EPTC, esprocarb, ethalfluralin, ethametsulphuron (-methyl), ethofumesate, ethoxyfen, ethoxysulphuron, etobenzanid, fenoxaprop (—P-ethyl), fentrazamide, flamprop (-isopropyl, -isopropyl-L, -methyl), flazasulphuron, florasulam, fluazifop (—P-butyl), fluazolate, flucarbazone (-sodium), flufenacet, flufenpyr, flumetsulam, flumiclorac (-pentyl), flumioxazin, flumipropyn, flumetsulam, fluometuron, fluorochloridone, fluoroglycofen (-ethyl), flupoxam, flupropacil, flupyrsulphuron (-methyl, -sodium), flurenol (-butyl), fluridone, fluroxypyr (-butoxypropyl, -meptyl), flurprimidol, flurtamone, fluthiacet (-methyl), fluthiamide, fomesafen, foramsulphuron, glufosinate (-ammonium), glyphosate (-isopropylammonium), halosafen, haloxyfop (-ethoxyethyl, —P-methyl), hexazinone, imazamethabenz (-methyl), imazamethapyr, imazamox, imazapic, imazapyr, imazaquin, imazethapyr, imazosulphuron, iodosulphuron (-methyl, -sodium), ioxynil, isopropalin, isoproturon, isouron, isoxaben, isoxachlortole, isoxaflutole, isoxapyrifop, ketospiradox, lactofen, lenacil, linuron, MCPA, mecoprop, mefenacet, mesotrione, metamitron, metazachlor, methabenzthiazuron, metobenzuron, metobromuron, (alpha-) metolachlor, metosulam, metoxuron, metribuzin, metsulphuron (-methyl), molinate, monolinuron, naproanilide, napropamide, neburon, nicosulphuron, norflurazon, orbencarb, oryzalihn, oxadiargyl, oxadiazon, oxasulphuron, oxaziclomefone, oxyfluorfen, paraquat, pelargon acid, pendimethalin, pendralin, penoxysulam, pentoxazone, penthoxamid, phenmedipham, picolinafen, piperophos, pretilachlor, primisulphuron (-methyl), profluazol, profoxydim, prometryn, propachlor, propanil, propaquizafop, propisochlor, procarbazone (-sodium), propyzamide, prosulphocarb, prosulphuron, pyraflufen (-ethyl), pyrazogyl, pyrazolate, pyrazosulphuron (-ethyl), pyrazoxyfen, pyribenzoxim, pyributicarb, pyridate, pyridatol, pyriftalid, pyriminobac (-methyl), pyrithiobac (-sodium), quinchlorac, quinmerac, quinoclamine, quizalofop (—P-ethyl, —P-tefuryl), rimsulphuron, sethoxydim, simazine, simetryn, sulcotrione, sulphentrazone, sulphometuron (-methyl), sulphosate, sulphosulphuron, tebutam, tebuthiuron, tepraloxydim, terbuthylazine, terbutryn, thenylchlor, thiafluamide, thiazopyr, thidiazimin, thifensulphuron (-methyl), thiobencarb, tiocarbazil, tralkoxydim, triallate, triasulphuron, tribenuron (-methyl), triclopyr, tridiphane, trifluralin, trifloxysulphuron, triflusulphuron (-methyl), tritosulphuron.

Furthermore suitable for the mixtures are known safeners, for example AD-67, BAS-145138, benoxacor, cloquintocet (-mexyl), cyometrinil, 2,4-D, DKA-24, dichlormid, dymron, fenclorim, fenchlorazol (-ethyl), flurazole, fluxofenim, furilazole, isoxadifen (-ethyl), MCPA, mecoprop (—P), mefenpyr (-diethyl), MG-191, oxabetrinil, PPG-1292, R-29148.

A mixture with other known active compounds, such as fungicides, insecticides, acaricides, nematicides, bird repellents, plant nutrients and agents which improve soil structure, is also possible.

The active compounds can be used as such, in the form of their formulations or in the use forms prepared therefrom by further dilution, such as ready-to-use solutions, suspensions, emulsions, powders, pastes and granules. They are used in a customary manner, for example by watering, spraying, atomizing or broadcasting.

The active compounds according to the invention can be applied both before and after emergence of the plants. They can also be incorporated into the soil before sowing.

The amount of active compound used can vary within a relatively wide range. It depends essentially on the nature of the desired effect. In general, the amounts used are between 1 g and 10 kg of active compound per hectare of soil surface, preferably between 5 g and 5 kg per ha.

As already mentioned above, it is possible to treat all plants and their parts according to the invention. In a preferred embodiment, wild plant species and plant cultivars, or those obtained by conventional biological breeding, such as crossing or protoplast fusion, and parts thereof, are treated. In a further preferred embodiment, transgenic plants and plant cultivars obtained by genetic engineering, if appropriate in combination with conventional methods (Genetically Modified Organisms), and parts thereof are treated. The term “parts” or “parts of plants” or “plant parts” has been explained above.

Particularly preferably, plants of the plant cultivars which are in each case commercially available or in use are treated according to the invention. Plant cultivars are to be understood as meaning plants having certain properties (“traits”) and which have been obtained by conventional breeding, by mutagenesis or by recombinant DNA techniques. They can be cultivars, bio- or genotypes.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions to be used according to the invention—also in combination with other agro-chemical active compounds, better crop plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products are possible which exceed the effects which were actually to be expected.

The transgenic plants or plant cultivars (i.e. those obtained by genetic engineering) which are preferably to be treated according to the invention include all plants which, in the genetic modification, received genetic material which imparted particularly advantageous useful properties (“traits”) to these plants. Examples of such properties are better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products. Further and particularly emphasized examples of such properties are a better defence of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, cotton, oilseed rape and also fruit plants (with the fruits apples, pears, citrus fruits and grapes), and particular emphasis is given to maize, soya beans, potatoes, cotton and oilseed rape. Traits that are emphasized are in particular the increased defence of the plants against insects by toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof) (hereinbelow referred to as “Bt plants”). Traits that are also particularly emphasized are the increased defence of the plants against fungi, bacteria and viruses by systemic acquired resistance (SAR), systemin, phytoalexins, elicitors and resistance genes and correspondingly expressed proteins and toxins. Traits that are furthermore particularly emphasized are the increased tolerance of the plants to certain herbicidally active compounds, for example imidazolinones, sulphonylureas, glyphosate or phosphinotricin (for example the “PAT” gene). The genes which impart the desired traits in question can also be present in combination with one another in the transgenic plants. Examples of “Bt plants” which may be mentioned are maize varieties, cotton varieties, soya bean varieties and potato varieties which are sold under the trade names YIELD GARD® (for example maize, cotton, soya bean), KnockOut® (for example maize), StarLink® (for example maize), Bollgard® (cotton), Nucotn® (cotton) and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya bean), Liberty Link® (tolerance to phosphinotricin, for example oilseed rape), IMI® (tolerance to imidazolinones) and STS® (tolerance to sulphonylurea, for example maize). Herbicide-resistant plants.(plants bred in a conventional manner for herbicide tolerance) which may be mentioned include the varieties sold under the name Clearfield® (for example maize). Of course, these statements also apply to plant cultivars having these genetic traits or genetic traits still to be developed, which plant cultivars will be developed and/or marketed in the future.

The plants listed can be treated according to the invention in a particularly advantageous manner with the compounds of the general formula I or the active compound mixtures according to the invention, where in addition to the good control of weed plants, the abovementioned synergistic effects with the transgenic plants or plant cultivars occur. The preferred ranges stated above for the active compounds or mixtures also apply to the treatment of these plants. Particular emphasis is given to the treatment of plants with the compounds or mixtures specifically mentioned in the present text.

The active compounds according to the invention are also suitable for controlling animal pests, in particular insects, arachnids and nematodes, found in agriculture, in forests, in the protection of stored products and materials and in the hygiene sector. They can preferably be used as crop protection compositions. They are active against normally sensitive and resistant species, and against all or individual developmental stages.

When used as insecticides, the active compounds according to the invention can furthermore be present in their commercially available formulations and in the use forms, prepared from these formulations, as a mixture with synergists. Synergists are compounds which increase the action of the active compounds, without it being necessary for the synergist added to be active itself.

The active compound content of the use forms prepared from the commercially available formulations can vary within wide limits. The active compound concentration of the use forms can be from 0.0000001 to 95% by weight of active compound, preferably between 0.0001 and 1% by weight.

The compounds are employed in a customary manner appropriate for the use forms.

The active compounds of the formula (I) according to the invention are also suitable for controlling arthropods which attack agricultural livestock such as, for example, cattle, sheep, goats, horses, pigs, donkeys, camels, buffaloes, rabbits, chickens, turkeys, ducks, geese, honey-bees, other domestic animals such as, for example, dogs, cats, caged birds, aquarium fish and so-called experimental animals such as, for example, hamsters, guinea pigs, rats and mice. By controlling these arthropods, cases of death and reductions in productivity (for meat, milk, wool, hides, eggs, honey and the like) should be diminished, so that more economical and simpler animal husbandry is possible by the use of the active compounds according to the invention.

The active compounds according to the invention are used in the veterinary sector in a known manner by enteral administration in the form of, for example, tablets, capsules, potions, drenches, granules, pastes, boluses, the feed-through method, suppositories, by parenteral administration such as, for example, by injections (intramuscularly, subcutaneously, intravenously, intraperitoneally and the like), implants, by nasal administration, by dermal administration in the form of, for example, immersing or dipping, spraying, pouring-on, spotting-on, washing, dusting, and with the aid of active-compound-comprising moulded articles such as collars, ear tags, tail tags, limb bands, halters, marking devices and the like.

When used for cattle, poultry, domestic animals and the like, the active compounds of the formula (I) can be applied as formulations (for example powders, emulsions, flowables) comprising the active compounds in an amount of 1 to 80% by weight, either directly or after 100- to 10 000-fold dilution, or they may be used as a chemical dip.

The active compounds are also suitable for controlling animal pests, in particular insects, arachnids and mites, which are found in enclosed spaces such as, for example, dwellings, factory halls, offices, vehicle cabins and the like. They can be employed on their own or in combination with other active compounds and excipients in domestic insecticide products for controlling these pests. They are active against sensitive and resistant species and against all developmental stages.

They are used as aerosols, pressureless spray products, for example pump and atomizer sprays, automatic fogging systems, foggers, foams, gels, evaporator products with evaporator tablets made of cellulose or polymer, liquid evaporators, gel and membrane evaporators, propeller-driven evaporators, energy-free, or passive, evaporation systems, moth papers, moth bags and moth gels, as granules or dusts, in baits for spreading or in bait stations.

The preparation and the use of the active compounds according to the invention is illustrated by the examples below.

### PREPARATION EXAMPLES

### Example 1

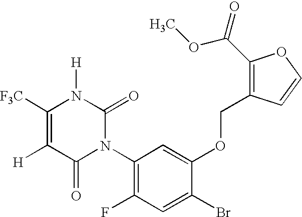

A mixture of 7.50 g (20.3 mMol) of 3-(4-bromo-2-fluoro-5-hydroxyphenyl)-6-tri-fluoromethyl-1H-pyrimidine-2,4-dione, 4.45 g (20.3 mMol) of methyl 3-bromomethylfuran-2-carboxylate, 5.6 g (40.6 mMol) of potassium carbonate and 120 ml of acetonitrile is heated under reflux for 6 hours. After cooling to room temperature, the mixture is poured into about the same amount by volume of 2N hydrochloric acid and shaken with ethyl acetate. The organic phase is separated off, dried with sodium sulphate and filtered. The filtrate is concentrated under reduced pressure and the residue is worked up by column chromatography (silica gel, methylene chloride/ethyl acetate, Vol.: 95/5).

This gives 5.80 g (55% of theory) of methyl 3-[2-bromo-5-(2,6-dioxo-4-trifluoromethyl-3,6-dihydro-2H-pyrimidin-1-yl)-4-fluorophenoxymethyl]furan-2-carboxylate.

### Example 2

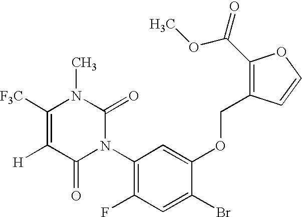

(Subsequent Reaction)

At room temperature (about 20° C.), a mixture of 2.50 g (4.93 mMol) of methyl 3-[2-bromo-5-(2,6-dioxo-4-trifluoromethyl-3,6-dihydro-2H-pyrimidin-1-yl)-4-fluorophenoxymethyl]furan-2-carboxylate, 0.82 g (5.92 mMol) of potassium carbonate and 65 ml of acetonitrile is stirred for 10 minutes, and a solution of 0.75 g (5.92 mMol) of dimethyl sulphate in 5 ml of acetonitrile is then added dropwise with stirring. The reaction mixture is then stirred at 45° C. for 2 hours and subsequently poured into about twice the amount of water and shaken with methylene chloride. The organic phase is separated off, dried with sodium sulphate and filtered. The filtrate is concentrated under reduced pressure, the residue is triturated with hexane and the resulting crystalline product is isolated by filtration with suction.

This gives 2.2 g (79% of theory) of methyl 3-[2-bromo-5-(2,6-dioxo-3-methyl-4-trifluoromethyl-3,6-dihydro-2H-pyrimidin-1-yl)-4-fluorophenoxymethyl]furan-2-carboxylate.

log P (pH 2.3)=3.34.

### Example 3

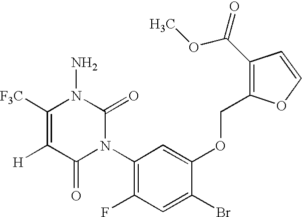

(Subsequent Reaction)

At room temperature (about 20° C.), a mixture of 2.50 g (4.93 mMol) of methyl 2-[2-bromo-5-(2,6-dioxo-4-trifluoromethyl-3,6-dihydro-2H-pyrimidin-1-yl)-4-fluorophenoxymethyl]furan-3-carboxylate, 0.50 g (5.92 mMol) of sodium bicarbonate and 50 ml of N,N-dimethylformarnmide is stirred for 10 minutes, and 1.18 g (5.92 mMol) of 1-aminooxy-2,4-dinitrobenzene are then added in little portions over a period of 6 hours. The reaction mixture is stirred at room temperature for 15 hours. A further 0.60 g of 1-aminooxy-2,4-dinitrobenzene is then added, and the mixture is stirred at room temperature for another 15 hours. The mixture is then poured into about twice the volume of water and then shaken with ethyl acetate. The organic phase is separated off, dried with sodium sulphate and filtered. The filtrate is concentrated under reduced pressure and the residue is worked up by column chromatography (silica gel, hexane/ethyl acetate, vol.: 2/1).

This gives 1.0 g (39% of theory) of methyl 2-[2-bromo-5-(3-amino-2,6-dioxo-4-trifluoromethyl-3,6-dihydro-2H-pyrimidin-1-yl)-4-fluorophenoxymethyl]furan-3-carboxylate.

log P (pH 2.3)=3.00.

Analogously to Examples 1 to 3, and in accordance with the general description of the preparation process according to the invention, it is also possible to prepare, for example, the compounds of the general formula (I) listed in Table 1 below.

TABLE 1
Examples of compounds of the formula (I)

| | |
|---|---|
| | (I) |
| 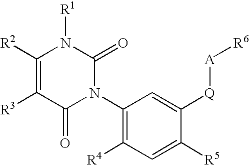 | |

| Ex. | | | | | | | | | |
|---|---|---|---|---|---|---|---|---|---|
| No. | A | Q | R¹ | R² | R³ | R⁴ | R⁵ | R⁶ | Physical data |
| 4 | CH₂ | O | CH₃ | CF₃ | H | F | CN | 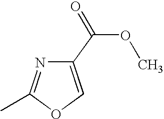 | |
| 5 | CH₂ | O | CH₃ | CF₃ | H | F | Br | 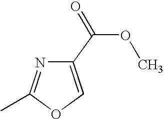 | logP = 2.77^{a)} |
| 6 | CH₂ | O | NH₂ | CF₃ | H | F | Br | 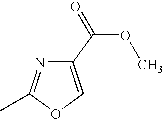 | logP = 2.47^{a)} |
| 7 | CH₂ | O | CH₃ | CF₃ | H | F | Br | 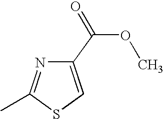 | logP = 2.97^{a)} |
| 8 | CH₂ | O | NH₂ | CF₃ | H | F | Br | 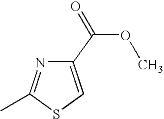 | logP = 2.68^{a)} |
| 9 | CH₂ | O | H | CF₃ | H | F | Cl | 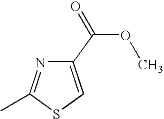 | |
| 10 | CH₂ | O | H | CF₃ | H | F | Cl | 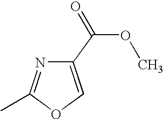 | |
| 11 | CH₂ | O | H | CF₃ | H | F | Br | 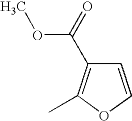 | logP = 2.73^{a)} |
| 12 | CH₂ | O | H | CF₃ | H | F | Br | 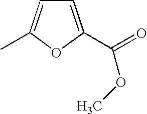 | logP =2.64^{a)} |
| 13 | CH₂ | O | H | CF₃ | H | F | Br | 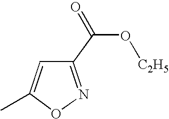 | |
| 14 | CH₂ | O | CH₃ | CF₃ | H | F | Br | 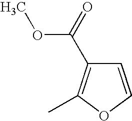 | logP = 3.32^{a)} |
| 15 | CH₂ | O | H | CF₃ | H | F | Br | 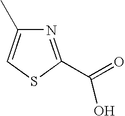 | |
| 16 | CH₂ | O | CH₃ | CF₃ | H | F | Br | 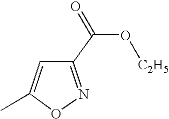 | logP = 3.34^{a)} |
| 17 | CH₂ | O | NH₂ | CF₃ | H | F | Br | 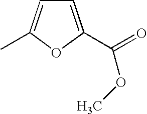 | logP = 2.88^{a)} |
| 18 | CH₂ | O | CH₃ | CF₃ | H | F | Br | 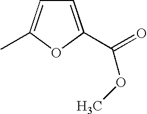 | logP = 3.20^{a)} |
| 19 | CH₂ | O | NH₂ | CF₃ | H | F | Cl | 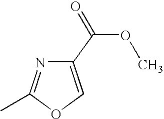 | logP = 2.39^{a)} |
| 20 | CH₂ | O | CH₃ | CF₃ | H | F | Cl | 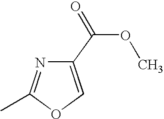 | logP = 2.67^{a)} |
| 21 | CH₂ | O | NH₂ | CF₃ | H | F | Cl | 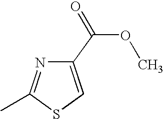 | logP = 2.61^{a)} |
| 22 | CH₂ | O | CH₃ | CF₃ | H | F | Cl | 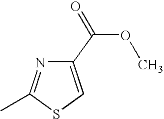 | logP = 2.90^{a)} |
| 23 | CH₂ | O | H | CF₃ | H | F | Cl | 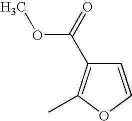 | |
| 24 | CH₂ | O | H | CF₃ | H | F | Cl | 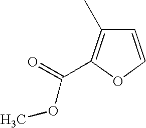 | logP = 2.67^{a)} |
| 25 | CH₂ | O | H | CF₃ | H | F | Cl | 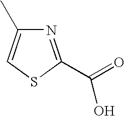 | |
| 26 | CH₂ | O | H | CF₃ | H | F | Cl | 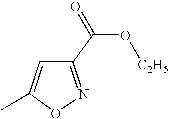 | logP = 2.73^{a)} |
| 27 | CH₂ | O | H | CF₃ | H | F | Cl | 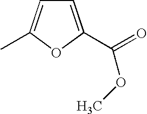 | logP = 2.58^{a)} |
| 28 | CH₂ | O | CH₃ | CF₃ | H | F | Br | 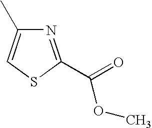 | logP = 3.09^{a)} |
| 29 | CH₂ | O | CH₃ | CF₃ | H | F | Cl | 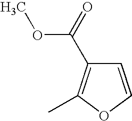 | logP = 3.27^{a)} |
| 30 | CH₂ | O | NH₂ | CF₃ | H | F | Cl | 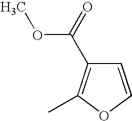 | logP = 2.94^{a)} |
| 31 | CH₂ | O | CH₃ | CF₃ | H | F | Cl | 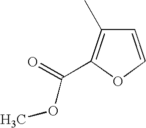 | log = 3.26^{a)} |
| 32 | CH₂ | O | NH₂ | CF₃ | H | F | Cl | 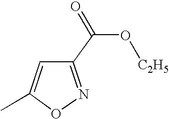 | logP = 2.98^{a)} |
| 33 | CH₂ | O | CH₃ | CF₃ | H | F | Cl | 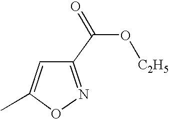 | logP = 3.28^{a)} |
| 34 | CH₂ | O | NH₂ | CF₃ | H | F | Cl | 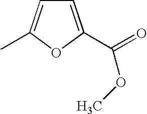 | logP = 2.82^{a)} |
| 35 | CH₂ | O | CH₃ | CF₃ | H | F | Cl | 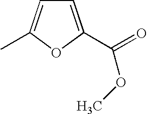 | |
| 36 | CH₂ | O | CH₃ | CF₃ | H | F | Cl | 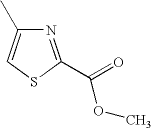 | logP = 3.00^{a)} |
| 37 | CH₂ | O | CH₃ | CF₃ | H | F | CN | 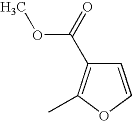 | logP = 2.91^{a)} |
| 38 | CH₂ | O | CH₃ | CF₃ | H | F | CN | 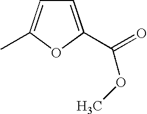 | logP = 2.79^{a)} |
| 39 | CH₂ | O | CH₃ | CF₃ | H | F | CN | 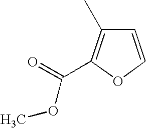 | logP = 2.89^{a)} |
| 40 | CH₂ | O | CH₃ | CF₃ | H | F | Cl | 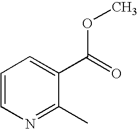 | |
| 41 | CH₂ | O | CH₃ | CF₃ | H | F | Br | 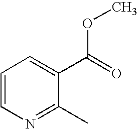 | logP = 1.62^{a)} |
| 42 | CH₂ | O | CH₃ | CF₃ | H | F | CN | 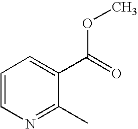 | |
| 43 | CH₂ | O | H | CF₃ | H | F | Cl | 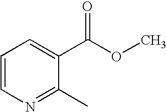 | |
| 44 | CH₂ | O | NH₂ | CF₃ | H | F | Cl | 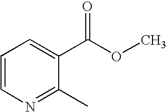 | |
| 45 | CH₂ | O | H | CF₃ | H | F | Br | 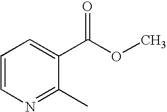 | |
| 46 | CH₂ | O | NH₂ | CF₃ | H | F | Br | 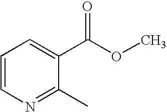 | logP = 2.61^{a)} |
| 47 | CH₂ | O | H | CF₃ | H | F | CN | 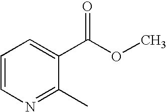 | |
| 48 | CH₂ | O | NH₂ | CF₃ | H | F | CN | 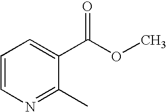 | |
| 49 | CH₂ | O | H | CF₃ | H | F | Cl | 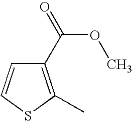 | |
| 50 | CH₂ | O | CH₃ | CF₃ | H | F | Cl | 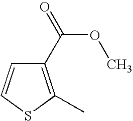 | logP = 3.71^{a)} |
| 51 | CH₂ | O | NH₂ | CF₃ | H | F | Cl | 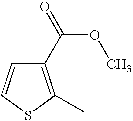 | logP = 3.36^{a)} |
| 52 | CH₂ | O | H | CF₃ | H | F | Br | 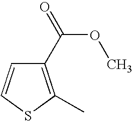 | |
| 53 | CH₂ | O | CH₃ | CF₃ | H | F | Br | 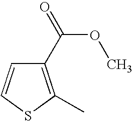 | logP = 3.80^{a)} |
| 54 | CH₂ | O | NH₂ | CF₃ | H | F | Br | 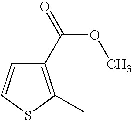 | logP = 3.44^{a)} |
| 55 | CH₂ | O | H | CF₃ | H | F | Cl | 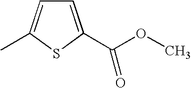 | |
| 56 | CH₂ | O | CH₃ | CF₃ | H | F | Cl | 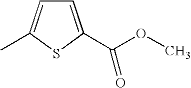 | logP = 4.45^{a)} |
| 57 | CH₂ | O | NH₂ | CF₃ | H | F | Cl | 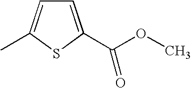 | logP = 3.13^{a)} |
| 58 | CH₂ | O | H | CF₃ | H | F | Br | 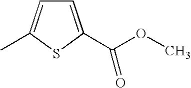 | |
| 59 | CH₂ | O | CH₃ | CF₃ | H | F | Br | 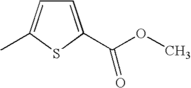 | logP = 3.52^{a)} |
| 60 | CH₂ | O | NH₂ | CF₃ | H | F | Br | 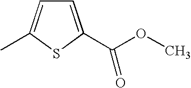 | |

The log P values given in the table were determined in accordance with EEC Directive 79/831 Annex V.A8 by HPLC (High Performance Liquid Chromatography) using a reversed-phase column (C 18). Temperature: 43° C.

(a) Mobile phases for the determination in the acidic range: 0.1% aqueous phosphoric acetonitrile; linear gradient from 10% acetonitrile to 90% acetonitrile—the sponding measurement results in Table 1 are marked ^{a)}.

(b) Mobile phases for the determination in the neutral range: 0.01 molar aqueous phosphate buffer solution, acetonitrile; linear gradient from 10% acetonitrile to 90% acetonitrile—the corresponding measurement results in Table 1 are marked^{b)}.

Calibration was carried out using unbranched alkan-2-ones (with 3 to 16 carbon atoms) with known log P values (determination of the log P values by the retention times using linear interpolation between two successive alkanones).

The lambda max values were determined in the maxima of the chromatographic signals using the UV spectra from 200 nm to 400 nm.

Starting Materials of the Formula (III):

### EXAMPLE III-1

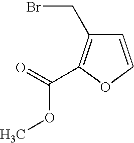

A mixture of 26.0 g (185.5 mMol) of methyl 3-methylfuran-2-carboxylate, 33.0 g (185.5 mMol) of N-bromosuccinimide (NBS), a spatula tip of 2,2′-azobis-2-methylpropanenitrile and 150 ml of carbon tetrachloride is heated under reflux for 15 hours and then filtered. The filtrate is worked up by distillation under reduced pressure.

This gives 24.7 g (50% of theory) of methyl 3-bromomethylfuran-2-carboxylate.

Boiling range: 62-64° C. (0.1 torr).

### USE EXAMPLES

### Example A

Pre-emergence test

Solvent: 5 parts by weight of acetone

Emulsifier: 1 part by weight of alkylaryl polyglycol ether

To produce a suitable preparation of active compound, 1 part by weight of active compound is mixed with the stated amount of solvent, the stated amount of emulsifier is added and the concentrate is diluted with water to the desired concentration.

Seeds of the test plants are sown in normal soil. After 24 hours, the soil is sprayed with the preparation of active compound such that the particular amount of active compound desired is applied per unit area. The concentration of active compound in the spray liquor is chosen such that the particular amount of active compound desired is applied in 1000 litres of water per hectare.

After three weeks, the degree of damage to the plants is rated in % damage in comparison to the development of the untreated control. The figures denote:
0%=no effect (like untreated control)
100%=total destruction

In this test, for example, the compounds of Preparation Examples 2, 3, 5, 6, 14, 17, 18, 19, 28, 29, 30, 31, 34, 35 and 36 exhibit very strong activity against weeds, and some of them are tolerated well by crop plants, such as, for example, maize, soya bean and wheat.

### Example B

Post-Emergence Test

Solvent: 5 parts by weight of acetone

Emulsifier: 1 part by weight of alkylaryl polyglycol ether

To produce a suitable preparation of active compound, 1 part by weight of active compound is mixed with the stated amount of solvent, the stated amount of emulsifier is added and the concentrate is diluted with water to the desired concentration.

Test plants with a height of 5-15 cm are sprayed with the preparation of active compound such that the particular amounts of active compound desired are applied per unit area. The concentration of the spray liquor is chosen such that the particular amounts of active compound desired are applied in 1000 l of water/ha.

After three weeks, the degree of damage to the plants is rated in % damage in comparison to the development of the untreated control. The figures denote:
0%=no effect (like untreated control)
100%=total destruction

In this test, for example, the compounds of Preparation Examples 2, 3, 4, 5, 6, 7, 8, 14, 16, 17, 18, 19, 20, 21, 22, 28, 29, 30, 31, 32, 33, 34, 35 and 36 exhibit very strong activity against weeds, and some of them are tolerated well by crop plants, such as, for example, wheat and sugar beet.

1-9. (canceled)

10. A compound of formula (I) 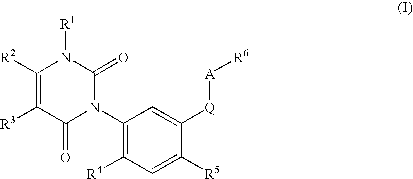
and the stereoisomeric forms thereof,
in which
A represents alkanediyl having 1 to 6 carbon atoms, alkenediyl having 2 to 6 carbon atoms, or alkynediyl having 2 to 6 carbon atoms,
Q represents O, S, SO, or SO₂,
R¹ represents hydrogen, amino, or optionally cyano-, halogen-, or C₁-C₄-alkoxy-substituted alkyl having 1 to 6 carbon atoms,
R² represents carboxyl, cyano, carbamoyl, or thiocarbamoyl; or represents optionally cyano-, halogen-, or C₁-C₄-alkoxy-substituted alkyl or alkoxycarbonyl having in each case 1 to 6 carbon atoms in the alkyl groups,
R³ represents hydrogen, halogen, or optionally halogen-substituted alkyl having 1 to 6 carbon atoms,
R⁴ represents hydrogen, cyano, carbamoyl, thiocarbamoyl, or halogen,
R⁵ represents nitro, cyano, carbamoyl, thiocarbamoyl, or halogen; or represents optionally halogen-substituted alkyl or alkoxy having in each case 1 to 6 carbon atoms, and
R⁶ represents monocyclic or bicyclic heterocyclyl having up to 9 carbon atoms and up to 5 heteroatoms consisting of up to 5 nitrogen atoms and/or up to 2 oxygen atoms and/or up to 2 sulphur atoms and optionally additionally up to 2 SO groups, up to 2 SO₂ groups, up to 2 CO groups, or up to 2 CS groups, wherein the heterocyclyl is attached via a carbon atom to A and is substituted by cyano, carboxyl, carbamoyl, or thiocarbamoyl, or substituted by C₁-C₆-alkoxycarbonyl, C₃-C₆-cycloalkyloxycarbonyl, or C₁-C₆-alkylaminocarbonyl, each of which is optionally substituted by cyano, carboxyl, halogen, or C₁-C₆-alkoxycarbonyl, or substituted by di(C₁-C₄-alkyl)aminocarbonyl or N—(C₁-C₄-alkoxy)-C₁-C₄-alkylaminocarbonyl, or substituted by C₂-C₆-alkenyloxycarbonyl or C₂-C₆-alkynyloxycarbonyl, each of which is optionally substituted by halogen, or substituted by cyano-C₁-C₆-alkyl, carboxy-C₁-C₆-alkyl, C₁-C₄-alkoxycarbonyl-C₁-C₆-alkyl, cyano-C₂-C₆-alkenyl, carboxy-C₂-C₆-alkenyl, or C₁-C₄-alkoxycarbonyl-C₂-C₆-alkenyl, each of which is optionally substituted by halogen, and optionally additionally substituted by nitro or halogen or by C₁-C₄-alkyl, C₁-C₄-alkoxy, C₁-C₄-alkylthio, C₁-C₄-alkylsulphinyl, or C₁-C₄-alkyl-sulphonyl, each of which is optionally substituted by halogen.

11. A compound of formula (I) according to claim 10 in which
A represents alkanediyl having 1 to 5 carbon atoms, alkenediyl having 2 to 5 carbon atoms, or alkynediyl having 2 to 5 carbon atoms,
Q represents O, S, or SO₂,
R¹ represents hydrogen, amino, or optionally cyano-, halogen-, or C₁-C₄-alkoxy-substituted alkyl having 1 to 4 carbon atoms,
R² represents carboxyl, cyano, carbamoyl, or thiocarbamoyl; or represents optionally cyano-, halogen-, or C₁-C₄-alkoxy-substituted alkyl or alkoxycarbonyl having in each case 1 to 4 carbon atoms in the alkyl groups,
R³ represents hydrogen, halogen, or optionally halogen-substituted alkyl having 1 to 4 carbon atoms,
R⁴ represents hydrogen, cyano, carbamoyl, thiocarbamoyl, fluorine, chlorine, bromine, or iodine,
R⁵ represents nitro, cyano, carbamoyl, thiocarbamoyl, or halogen; or represents optionally halogen-substituted alkyl or alkoxy having in each case 1 to 4 carbon atoms, and
R⁶ represents monocyclic or bicyclic heterocyclyl having up to 9 carbon atoms and up to 5 heteroatoms consisting of up to 3 nitrogen atoms and/or one oxygen atom and/or one sulphur atom, and optionally additionally one —SO group, one —SO₂ group, one —CO group, or one —CS group, wherein the heterocyclyl is attached via a carbon atom to A and is substituted by cyano, carboxyl, carbamoyl, or thiocarbamoyl, or substituted by C₁-C₅-alkoxycarbonyl, C₅-C₆-cycloalkyloxycarbonyl, or C₁-C₅-alkylaminocarbonyl, each of which is optionally substituted by cyano, carboxyl, halogen, or C₁-C₄-alkoxycarbonyl, or substituted by di(C₁-C₄-alkyl)aminocarbonyl or N—(C₁-C₄-alkoxy)-C₁-C₄-alkylaminocarbonyl, or substituted by C₂-C₅-alkenyloxycarbonyl or C₂-C₅-alkynyloxycarbonyl, each of which is optionally substituted by halogen, or substituted by cyano-C₁-C₅-alkyl, carboxy-C₁-C₅-alkyl, C₁-C₄-alkoxy-carbonyl-C₁-C₅-alkyl, cyano-C₂-C₅-alkenyl, carboxy-C₂-C₅-alkenyl, or C₁-C₄-alkoxycarbonyl-C₂-C₅-alkenyl, each of which is optionally substituted by halogen, and optionally additionally substituted by nitro or halogen or by C₁-C₄-alkyl, C₁-C₄-alkoxy, C₁-C₄-alkylthio, C₁-C₄-alkylsulphinyl, or C₁-C₄-alkylsulphonyl, each of which is optionally substituted by halogen.

12. A compound of formula (I) according to claim 10 in which
A represents alkanediyl having 1 to 4 carbon atoms, alkenediyl having 2 to 4 carbon atoms, or alkynediyl having 2 to 4 carbon atoms,
Q represents O or S,
R¹ represents hydrogen, amino, or optionally cyano-, fluorine-, chlorine-, methoxy-, or ethoxy-substituted methyl, ethyl, or n- or i-propyl,
R² represents carboxyl, cyano, carbamoyl, or thiocarbamoyl; or represents optionally cyano-, fluorine-, chlorine-, methoxy-, ethoxy-, or n- or i-propoxy-substituted alkyl or alkoxycarbonyl having in each case 1 to 3 carbon atoms in the alkyl groups,
R³ represents hydrogen, fluorine, chlorine, bromine, or optionally fluorine- and/or chlorine-substituted methyl, ethyl, or n- or i-propyl,
R⁴ represents hydrogen, cyano, carbamoyl, thiocarbamoyl, fluorine, chlorine, or bromine,
R⁵ represents nitro, cyano, carbamoyl, thiocarbamoyl, fluorine, chlorine, or bromine; or represents optionally fluorine- and/or chlorine-substituted alkyl or alkoxy having in each case 1 to 3 carbon atoms, and
R⁶ represents heterocyclyl selected from the group consisting of furyl, benzofuryl, tetrahydrofuryl, thienyl, benzothienyl, pyrrolyl, benzopyrrolyl, pyrrolinyl, pyrazolyl, benzopyrazolyl, pyrazolinyl, imidazolyl, benzimidazolyl, imidazolinyl, oxazolyl, benzoxazolyl, oxazolinyl, isoxazolyl, isoxazolinyl, thiazolyl, benzthiazolyl, thiazolinyl, thiadiazolyl, triazolyl, pyridinyl, pyrimidinyl, and triazinyl, wherein the heterocyclyl is attached via a carbon atom to A and is substituted by cyano, carboxyl, carbamoyl, or thiocarbamoyl, or substituted by C₁-C₄-alkoxycarbonyl, C₅-C₆-cycloalkyloxycarbonyl, or C₁-C₄-alkylaminocarbonyl, each of which is optionally substituted by cyano, carboxyl, halogen, or C₁-C₄-alkoxycarbonyl, or substituted by di(C₁-C₃-alkyl)aminocarbonyl or N—(C₁-C₃-alkoxy)-C₁-C₃-alkylaminocarbonyl, or substituted by C₃-C₄-alkenyloxycarbonyl or C₃-C₄-alkynyloxycarbonyl, each of which is optionally substituted by halogen, or substituted by cyano-C₁-C₃-alkyl, carboxy-C₁-C₃-alkyl, C₁-C₄-alkoxycarbonyl-C₁-C₃-alkyl, cyano-C₂-C₃-alkenyl, carboxy-C₂-C₃-alkenyl, or C₁-C₄-alkoxycarbonyl-C₂-C₃-alkenyl, each of which is optionally substituted by halogen, and optionally additionally substituted by nitro or halogen or by C₁-C₄-alkyl, C₁-C₄-alkoxy, C₁-C₄-alkylthio, C₁-C₄-alkylsulphinyl, or C₁-C₄-alkylsulphonyl, each of which is optionally substituted by halogen.

13. A compound of formula (I) according to claim 10 in which
A represents methylene, ethane-1,1-diyl, ethane-1,2-diyl(dimethylene), propane-1,1-diyl, propane-1,2-diyl, propane-1,3-diyl, ethene-1,1-diyl, ethane-1,2-diyl, propene-1,1-diyl, propene-1,2-diyl, propene-1,3-diyl, ethyne-1,2-diyl, or propyne-1,3-diyl,
Q represents O,
R¹ represents hydrogen, amino, or optionally cyano-, fluorine-, chlorine-, methoxy-, or ethoxy-substituted methyl or ethyl,
R² represents carboxyl, cyano, carbamoyl, or thiocarbamoyl; or represents optionally cyano-, fluorine-, chlorine-, methoxy- or ethoxy-substituted methyl, ethyl, methoxycarbonyl, or ethoxycarbonyl,
R³ represents hydrogen, fluorine, chlorine, bromine, or optionally fluorine- and/or chlorine-substituted methyl or ethyl,
R⁴ represents hydrogen, cyano, fluorine, or chlorine,
R⁵ represents nitro, cyano, carbamoyl, thiocarbamoyl, fluorine, chlorine, bromine, or optionally fluorine- and/or chlorine-substituted methyl, ethyl, methoxy, or ethoxy, and
R⁶ represents heterocyclyl selected from the group consisting of furyl, thienyl, pyrrolyl, pyrrolinyl, pyrazolyl, pyrazolinyl, imidazolyl, imidazolinyl, oxazolyl, oxazolinyl, isoxazolyl, isoxazolinyl, thiazolyl, thiazolinyl, pyridinyl, and pyrimidinyl, wherein the heterocyclyl is attached via a carbon atom to A and is substituted by cyano, carboxyl, carbamoyl or thiocarbamoyl, or substituted by methoxycarbonyl, ethoxycarbonyl, n- or i-propoxycarbonyl, cyclopentyloxycarbonyl, cyclohexyloxycarbonyl, methylaminocarbonyl, ethylaminocarbonyl, or n- or i-propylaminocarbonyl, each of which is optionally substituted by cyano, carboxyl, fluorine, chlorine, methoxycarbonyl, ethoxycarbonyl, or n- or i-propoxycarbonyl), or substituted by dimethylaminocarbonyl, diethylaminocarbonyl, or N-methoxymethylaminocarbonyl, or substituted by propenyloxycarbonyl, butenyloxycarbonyl, propynyloxycarbonyl, or butynyloxycarbonyl, each of which is optionally substituted by fluorine or chlorine, or substituted by cyanomethyl, cyanoethyl, cyanopropyl, carboxymethyl, carboxyethyl, carboxypropyl, methoxycarbonylmethyl, ethoxycarbonylmethyl, n- or i-propoxycarbonylmethyl, methoxycarbonylethyl, ethoxycarbonylethyl, n- or i-propoxycarbonylmethyl, cyanoethenyl, cyanopropenyl, carboxyethenyl, carboxypropenyl, methoxycarbonylethenyl, ethoxycarbonylethenyl, n- or i-propoxycarbonylethenyl, methoxycarbonylpropenyl, ethoxycarbonylpropenyl, or n- or i-propoxycarbonylpropenyl, each of which is optionally substituted by fluorine and/or chlorine, and optionally additionally substituted by nitro, fluorine, chlorine, bromine, or by methyl, ethyl, n- or i-propyl, n-, i-, s-, or t-butyl, methoxy, ethoxy, n- or i-propoxy, methylthio, ethylthio, n- or i-propylthio, methylsulphinyl, ethylsulphinyl, methylsulphonyl, or ethylsulphonyl, each of which is optionally substituted by fluorine and/or chlorine.

14. A compound of formula (I) according to claim 10 in which
A represents methylene,
R¹ represents hydrogen, methyl, or amino,
R² represents trifluoromethyl,
R³ represents hydrogen,
R⁴ represents fluorine,
R⁵ represents chlorine, bromine, or cyano, and
R⁶ represents one of the groups (A) to (L) 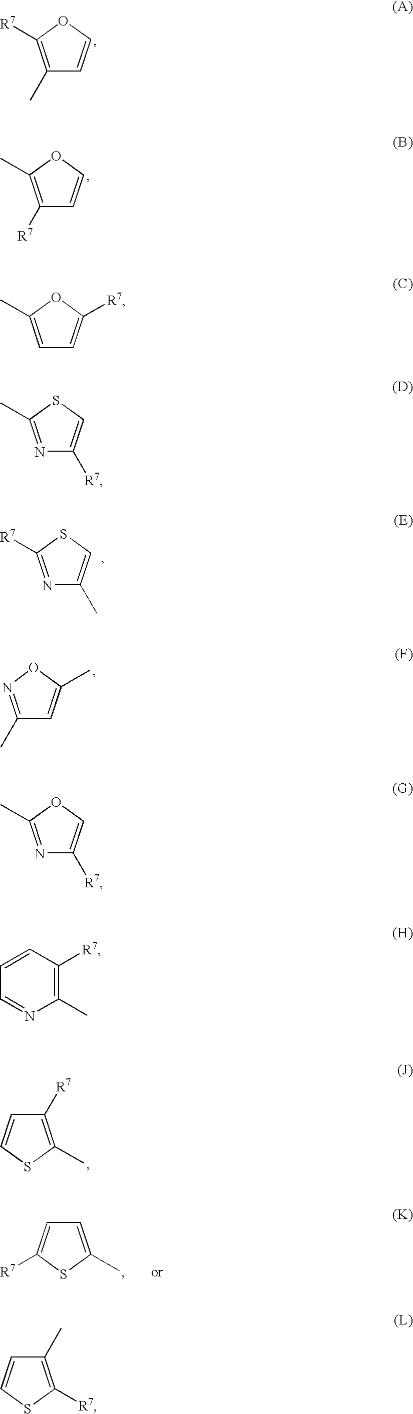
in which R⁷ represents carboxyl, carbamoyl, methoxycarbonyl, ethoxycarbonyl, or n- or i-propoxycarbonyl.

15. A process for preparing compounds of formula (I) according to claim 10 comprising reacting a compound of formula (II) 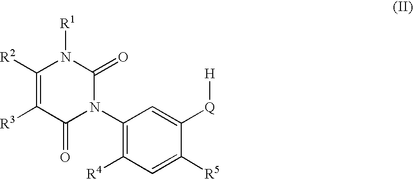
in which Q, R¹, R², R³, R⁴, and R⁵ are as defined for formula (I) in claim 10, with a substituted heterocycle of formula (III) 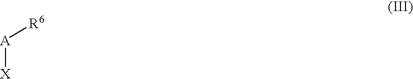
in which
A and R⁶ are as defined for formula (I) in claim 10, and
X represents halogen or represents optionally substituted alkylsulphonyloxy or arylsulphonyloxy,
optionally in the presence of one or more reaction auxiliaries and optionally in the presence of one or more diluents, to form a compound of formula (I).

16. A process according to claim 15 additionally comprising converting the compound of formula (I) into one or more other compounds of formula (I).

17. A composition comprising one or more compounds according to claim 10 and one or more extenders.

18. A method for controlling unwanted plants and/or arthropods comprising allowing an effective amount of one or more compounds of formula (I) according to claim 10 to act on the plants and/or arthropods and/or their habitat.

19. A method for controlling unwanted plants and/or arthropods comprising allowing an effective amount of a composition according to claim 17 to act on the plants and/or arthropods and/or their habitat.
